(19) United States
(12) Patent Application Publication
(10) Pub. No.: US 2004/0219694 A1
(43) Pub. Date: Nov. 4, 2004

(54) Lateral flow test format for enzyme assays

(76) Inventors: Roger Steward Chittock, Darmstadt (DE); Ian David Watkins, Darmstadt (DE)

Correspondence Address: Millen White, Zelano & Branigan, Arlington Courthouse Plaza I, 2200 Clarendon Boulevard Suite 1400, Arlington, VA, 22201, US

(21) Appl. No.: 10/489,166

(22) PCT Filed: Aug. 1, 2002
(86) PCT No.: PCT/EP02/08575

(30) Foreign Application Priority Data
Sep. 11, 2001 (EP) 011218203

(51) Int. Cl. G01N 33/543

(52) U.S. Cl. 436/518

(57) ABSTRACT

The invention relates to a lateral flow enzyme assay device and test kit for the determination of analyte in a test sample. The invention further relates to lateral flow method for the determination of analytes by directly using the analyte as an enzyme substrate.

### FIELD OF THE INVENTION

The invention relates to a lateral flow enzyme assay device and test kit for the determination of an analyte in a test sample. The invention further relates to a lateral flow method for the detection of analytes by using the analyte as an enzyme substrate or co-substrate.

### BACKGROUND OF THE INVENTION

Many analyses of biochemicals are based on enzyme assays. A wide variety of assays are used in the fields of clinical chemistry, hygiene monitoring, life sciences research including molecular biology, microbiology and water testing.

Typically the analyte of interest is a substrate or co-substrate for an enzyme-catalysed reaction that, with addition of further substrate components, yields a product more easily detected and measured than the original analyte. Such assays can involve single or multiple enzyme steps. Detection methods can include an increase in absorbance, particularly a change in colour, change in fluorescence, change in luminescence, change in electrical potential at a surface, change in other optical properties such as circular dichroism or light scattering, or any other easily measured physical property.

Most laboratory based tests for qualitative or quantitative detection of an analyte use formats in which precise amounts of two or more liquid reagents are added to the sample according to a defined protocol to produce a signal more easily detectable than direct measurement of the analyte.

For a typical enzyme assay procedure, the operator needs enzyme and substrate reaction components to react with the analyte in solution in the sample. In addition, there may be a need for buffering components, agents to extract the analyte of interest from the sample matrix further enzyme components, further enzyme co-factors, additional signal components and a known standard concentration of analyte as a positive control for comparison with the unknown level in the sample.

Due to unwanted chemical cross-reactions and/or inappropriate ionic conditions and/or effects leading to instability in storage, the different assay components are often incompatible with one another. As a result, the assay components need to be formulated into two or more separate reagents. It is therefore usual to have at least separate enzyme and substrate reagents to add to the sample in order to undertake an enzyme assay.

As a consequence, experimental protocols for enzyme assays are often lengthy and complicated, with extensive preparation steps before the assay can be performed and the requirement for specialised apparatus and a high level of skill and training on the part of the operator. Liquid based assays require extra reaction vessels such as tubes, a means to accurately dispense solutions such as a micro-pipette and a means to dispose of the reaction components safely after use.

In addition it is often difficult to stabilise enzymes in solution within liquid reagents. Freeze dried reagents have improved stability but the need to supply one or more solutions for rehydration and also the extra steps involved represent a significant decrease in user convenience.

A new high-convenience format or device for enzyme assays would give significant benefits if it were to feature the following aspects:

Easy handling without the need for lengthy and complicated preparation steps.

The capability to incorporate from one to several assay reagents in stable formulations.

The potential to be read by a suitable instrument

The incorporation of a positive control within the same device.

Ease of manufacture.

Robust construction, enabling field use.

Such a device would be highly suitable for unskilled operators and for use both in and outside a laboratory environment.

A substantial improvement has been achieved in the last years in the field of immunoassays. Lateral flow assays in which reagents and samples are transported within a linear matrix containing reagents to detect the presence of one or more specific analytes, fulfil the above mentioned requirements. Lateral flow assays typically are immunoassays in which analytes are identified by the binding of specific antibodies. Most assays rely on two binding events, with analyte identification resulting in the analyte moving from the mobile to the immobile phase of the assay. The first binding reaction occurs in the mobile phase between the analyte and a specific binding molecule for the analyte with an attached label. The second binding reaction occurs between the analyte-binding molecule-label complex and a second specific binding molecule immobilised at the reaction site. The two binding reactions combined concentrate the label at the reaction site if the analyte is present, forming the basis of analyte detection.

For example U.S. Pat. No. 5,591,645 discloses a method and a device for determining the presence of an analyte in a liquid sample comprising an immobilised and a mobile analyte binding agent.

A similar method and device is disclosed by U.S. Pat. No. 5,770,460 in which the detection of analyte relies on the binding of an analyte binding agent with an attached visible label to the analyte in the mobile phase before capture by an immobile analyte binding agent.

Lateral flow assays have been described, for example WO97/09620, in which an analyte binding agent with a covalently-linked enzyme binds the analyte which in turn binds to a non-diffusibly attached analyte receptor, thereby immobilising the enzyme if analyte is present.

U.S. Pat. No. 5,710,005 includes several examples of binding reactions to indefinitely immobilise one or more components present in the mobile phase that can be used in addition to antibody binding reactions for lateral flow assays.

A critical step in analyte detection of the prior art lateral flow assays is the localisation of the label from the mobile phase to the immobile phase, as an indirect result of the binding interactions of the binding agents, to one of which the enzyme or other label is linked.

It has now been found that it is possible to further simplify the lateral flow test format and to use it also for enzyme assays in which no labeling or permanent binding of the analyte takes place.

### SUMMARY OF THE INVENTION

The present invention relates to a method for determining the presence of an analyte in a sample comprising:

a) providing a test device incorporating a lateral flow test strip comprising a dry matrix material capable of transporting a liquid therealong by capillarity and having at least a start zone for receiving said sample and a reaction zone having at least one kind of enzyme immoblilized therein;

b) contacting the start zone with said sample thereby causing a liquid phase to move through said test strip;

c) determining a detectable signal at the reaction zone directly or indirectly caused by covalent modification of the analyte by the enzymes at the reaction zone.

In one preferred embodiment the test strip provided in step a) comprises an additional reagent zone.

In one embodiment the test strip provided in step a) comprises additional dried reagents in the start zone and/or the reagent zone and/or the reaction zone.

In another embodiment the liquid flow generated in step b) is enhanced by a wick on the test strip.

In a preferred embodiment the detectable signal is generated by a product of an enzymatic reaction which is visible by eye and barely soluble.

In a preferred embodiment the detectable signal in step c) is generated by using a recycling enzyme reaction.

The present invention further relates to a lateral flow test device for determining the presence of an analyte in a sample at least incorporating a test strip comprising a dry matrix material capable of transporting a liquid therealong by capillarity and having at least a start zone for receiving said sample and a reaction zone having at least one kind of enzyme immoblilized therein whereby the enzymes and optional additional reagents on the test strip are suitable for covalently modifying the analyte and thus directly or indirectly generating a detectable signal.

In a preferred embodiment the test strip further comprises a solid support on which is attached the dry matrix material.

In a preferred embodiment the test strip further comprises a reagent zone containing dried reagents.

In a very preferred embodiment the test strip comprises a start zone containing a dried detergent extractant, a reagent zone containing one or more dried enzyme substrates and/or co-factors and a reaction zone containing at least one kind of immobilised enzyme.

In another preferred embodiment the test strip further comprises a wick.

In another embodiment the test device further comprises a housing.

In a very preferred embodiment the lateral flow test strip comprises a start zone containing DTAB (Dodecyl Trimethyl Ammonium Bromide) as detergent extractant, a reagent zone containing the substrates NBT and glucose and a reaction zone containing the immobilised enzymes glucose dehydrogenase and diaphorase.

The present invention further relates to the use of the test device according to the invention in an enzyme assay.

In a preferred embodiment the test device is used to test surfaces.

In another preferred embodiment the test device is used to test liquids.

The present invention further relates to an enzyme assay test kit comprising a lateral flow test device according to the present invention.

### BRIEF DESCRIPTION OF THE DRAWINGS

FIG. 1 is a scheme of an enzymatic reaction that may be utilised in the present invention.

FIG. 2 is a schematic view of a lateral flow test device according to the present invention.

### DETAILED DESCRIPTION OF THE INVENTION

The basis of the present invention is the finding that the lateral flow principle can also be used for enzyme assays. In lateral flow immunoassays according to the state of the art the specificity for an analyte is generated by specific binding interaction between the analyte and the antigen recognition domains of the antibody molecules used, said binding being effectively permanent in relation to the time course of the assay. This is analogous to ligand-receptor binding. Two distinct reagents are required:

1. Unlabelled antibody molecules immobilised on the lateral flow immunoassay device serve the purpose of capturing the analyte at a defined location on the device.

2. In so-called sandwich assay formats, the second reagent comprises antibody molecules, which are not immobilised to the device, with attached label(s). These molecules are also bound to the analyte during the immunoassay. In so-called competitive assay formats, the second reagent comprises labelled tracer ligand molecules which can also be bound by the same recognition domains of the immobilised antibody capture molecules as the analyte, so that the amount of said tracer that can be bound is inversely proportional to the amount of analyte present in the sample.

Assay of the analyte is realised in either case by detecting the label at the location of the capture. The analyte itself does not contribute to the signal for detection of the label.

According to the state of the art alternative molecules can be used in the place of antibodies, provided such molecules provide specific and permanent binding of the analyte.

Conversely, in the enzyme assays according to the present invention, the signal for detection of the analyte is not realised by a label. The analyte is not permanently bound to an antibody, a receptor or to any other molecule. It is detected rather by its transient participation in an enzyme reaction. During this enzyme reaction the analyte is chemically modified and thus directly or indirectly participates in the reaction that creates a detectable signal. As a consequence, in the enzyme assays according to the invention, specificity for the analyte is provided by an enzyme which uses the analyte as a substrate, co-substrate or co-factor and chemically modifies it and the signal for detection and measurement of the analyte is thus dependent upon the turnover of the enzyme-mediated reaction involving the analyte.

The lateral flow enzyme assay according to the present invention is performed with a lateral flow test device comprising at least a lateral flow test strip. This test strip comprises at least a dry matrix material capable of transporting a liquid therealong by capillarity with a start zone for receiving a sample and a reaction zone in which the enzyme reaction takes place which is specific for the analyte and which generates a detectable signal.

The dry matrix material is typically fixed to a solid support in form of one or more absorbent interconnecting porous pads. The matrix material is a typically a bibulous material such as conventional cellulosic paper, nitrocellulose or derivatives thereof which may be have been chemically treated to enhance assay characteristics, such as hydrophilic properties. The matrix material should be compatible with the stability of the relevant assay reagents, the chemistry of the assay and with required lateral flow characteristics such as flow rate. The solid support is typically non-water absorbent material, often plastic such as polyester sheet. The support is typically bonded to the dry matrix materials by adhesive. All such materials above are well known to those skilled in the art.

The start zone is preferably situated at one end of the test strip. The sample to be analysed is applied to the start zone. In a preferred embodiment, the start zone is a sample pad composed of an absorbent paper. Glass fibre materials, fibrous plastic materials such as Porex® sheet materials (Porex Corporation), and non-woven fabrics comprising such materials as viscose and polyester may also be used. Preferably such materials should be in a single sheet or layer. The volume capacity of the sample pad defines the initial sample uptake of the device. The sample pad also acts as a filter to help to prevent unwanted particulate materials from reaching the reaction zone. The material of the start zone helps to control the release of any impregnated mobilisable components of the assay to the rest of the device and should therefore be compatible with this purpose.

Liquid samples can be added to the start zone as single drops e.g. by using a pipette or by dipping the start zone in the liquid sample. The start zone can comprise components which are dried onto it such as components of the enzyme assay and/or chemicals to prevent (“block”) non-specific binding effects at the reaction zone and/or to chemicals to enhance hydrophilic properties, rehydration of assay components and lateral flow characteristics and/or analyte release agents and/or extractants, preferably detergent extractants.

The reaction zone might be located directly adjacent to the start zone or further down the test strip. It contains one or more components of the enzyme assay in a stable state, in particular at least one kind of enzyme immobilised to the reaction zone. Additional components might be additional immobilised enzymes and/or immobilised or dried enzyme substrates, co-substrates co-factors etc. In a preferred embodiment the reaction zone is a pad or a membrane chosen to be suitable for procedures to non-covalently or covalently immobilise protein without denaturation and also for the lateral flow characteristics of the material. In a very preferred embodiment, the reaction zone is composed of a nitrocellulose membrane with a high protein binding capacity. Such membrane may also incorporate cellulose acetate. The pad or membrane may be treated before and/or after enzyme immobilisation in order to minimise unwanted effects such as non-specific binding of analyte and/or assay components and/or in order to enhance hydrophilic properties and lateral flow. It is on the reaction zone that the reactions comprising the assay occur and the product indicating the presence of the analyte in the original sample is formed.

In a preferred embodiment the test strip further comprises a reagent zone, typically composed of materials similar to those employed for the sample pad, preferably composed of absorbent paper or of glass fibre or of polyethylene fibre or of polyester, localised between the start zone and the reaction zone or partially overlapping with one or both of these zones. The reagent zone contains one or more components of the assay dried in stable state such as enzyme substrate components and/or chemicals to prevent (“block”) non-specific binding effects at the reaction zone and/or chemicals to enhance hydrophilic properties, rehydration of assay components and lateral flow characteristics and/or analyte release agents and/or extractants, preferably detergent extractants instead or in addition to the start zone. The material of the reagent zone helps to control the release of impregnated mobilisable components of the assay to the reaction zone and should therefore be compatible with this purpose.

In another preferred embodiment the test strip incorporates a wick or a comparable means which draws liquid sample from the matrix of the test strip through the device and therefore drives the capillary flow from the sample pad. Flow of solution into the wick ensures a sustained flow from the sample and reagent pads across the reaction site of the immobilised enzymes to maximise the amount of product formed. The wick is typically composed of materials similar to those employed for the sample pad. In a preferred embodiment, the wick is composed of an absorbent paper. The assay reaches endpoint only when the volume capacity of the absorbent pad is filled, assuming that sample volume is not limiting.

In one embodiment the test device further comprises a film made of plastic or other suitable material overlaying the reaction and/or the reagent zones of the device to prevent contamination of the device during operation while still allowing any signal produced by the device to be detected. For example a false positive result may occur with a device designed to detect NAD and NADH if the reaction zone is touched during operation due the natural occurrence of these compounds on the fingers. The employment of this film allows easy handling and operation without the risk of contamination.

In one embodiment the test device further comprises a plastic case or other housing to provide additional strength and rigidity to the device and to facilitate handling of the device without contaminating it. Preferably, the test strip is only partially covered by the housing. The start zone is preferably not covered by the housing to ensure easy application of the sample.

The various absorbent zones of the test strip are all in the same plane, allowing capillary flow of a liquid sample between the zones. In the process, any component(s) deployed in dried state on the test strip, such as detergent to extract the analyte from matrices within the sample, are reconstituted into solution. All zones may contain further reagents like stabilising agents or buffers which e.g. support the storage of the components of the enzyme reaction or provide reaction conditions (e.g. pH) that are suitable for the enzyme reaction. For some applications it might be favourable to combine the enzyme reaction with a chemical reaction to e.g. improve the generation of a detectable signal. In this case, the components of the chemical reaction are also included in dry state in the zones of the test strip.

In a preferred embodiment of the present invention, the substrates of the enzyme reaction are placed in the reagent pad because they typically have a low molecular weight and are mobile in solution. The enzymes are positioned on the reaction pad because they have a high molecular weight and are relatively immobile. In a very preferred embodiment the reaction pad is composed of a nitrocellulose membrane to which the enzymes will bind so tightly as to be effectively immobilised.

In order to adapt the test strip of the present invention to more complex determinations, multiple reagent zones can be incorporated into zone designs as required and/or multiple enzyme components can be deposited at different points on the reaction zone.

By the deployment of multiple components in this way, it is possible to have control reactions fully incorporated into the device of the invention. Such reactions can be used to assess that the device has functioned correctly and can be used in order to facilitate semi-quantitative comparison between a suitable control reaction and the amount of product generated by the assay for the analyte of interest.

One embodiment uses more than one enzyme or group of enzymes or other reagents capable of detecting more than one analyte of interest at separate locations on the test device.

One further embodiment is to incorporate both enzyme assay and immunoassay within the same test strip.

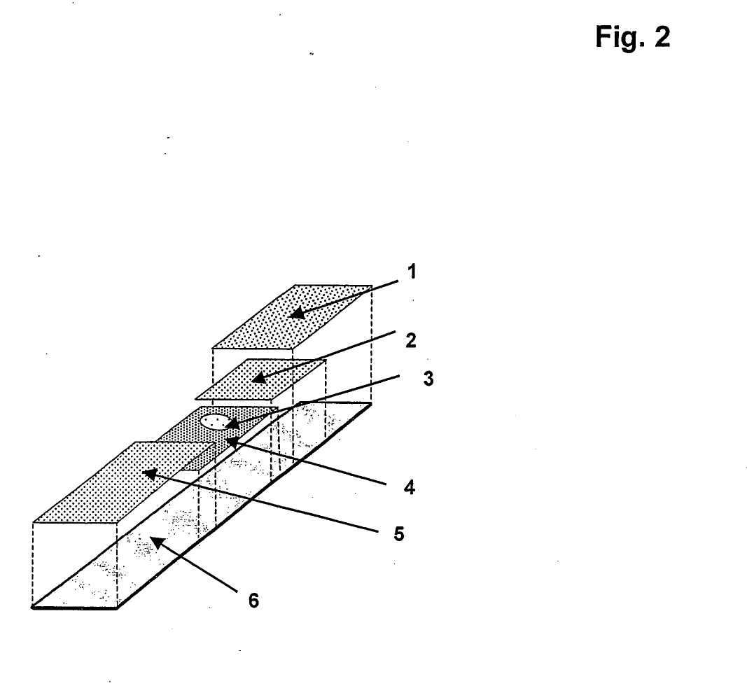

FIG. 2 is a schematic, illustrative view of a lateral flow test strip according to the present invention. On a self-adhesive plastic base (6) are located a sample pad as start zone (1), a reagent zone (2) partially overlapping with the start zone, a reaction zone (4) with enzyme (3) immobilised on to it. Adjacent to the reaction zone is a wick (5).

The method of the present invention for detecting an analyte in a sample by a lateral flow enzyme assay comprises

a) providing a lateral flow test device according to the present invention

b) applying a sample to the start zone of the test strip whereby capillarity draws liquid from the start zone to the other zones of the test strip

c) determining a detectable signal at the reaction zone

If a liquid sample is applied to the test strip the volume of the sample liquid is normally sufficient to ensure flow of the liquid through the test strip. The device may also be used to sample direct from a bulk fluid by bringing the start zone of the device into contact with said fluid in order to initiate capillary flow, activate the device and uptake a volume of sample according to the capacity of the device. In this respect the device may be described as “self-sampling.”

In case of dry or low-volume liquid samples, the application of the sample to the start zone and the generation of capillary flow can be facilitated by the use of a wetting agent. The wetting agent might be applied to the start zone before and/or after the application of the sample.

If the wetting agent is applied after the application of the sample, in a first step, a certain amount of a solid or low-volume liquid sample is applied on the test strip, e.g. by pressing the start zone of the test strip on the sample or by wiping the test strip over a sample area (e.g. in case of determination of surface cleanliness). In a second step an amount of a wetting agent suitable to allow liquid flow is applied to the start zone. Preferably, the wetting agent is water or a buffer solution. In either case, the wetting agent may also contain extractant such as detergent to release the analyte from within the sample.

In another version of this embodiment which is especially suitable for the sampling of surfaces, for example in the food preparation or manufacturing industry, a drop of wetting agent is applied to the start zone, then the surface is wiped. The wetting solution may contain an appropriate extractant such as detergent or similar agent to facilitate the transfer of analyte from the surface to the start zone and release analyte from the sample matrix into solution. The pressure applied to compress the sample pad of the start zone also initiates the capillary flow of liquid to the reagent pad and onwards.

In a more refined embodiment, the liquid capacity of the start zone is adjusted so that a defined volume of wetting solution, e.g. one drop or a defined number of drops, is sufficient to moisten the sample pad, but not sufficient to pass into the other device components to initiate the assay. After sampling a second volume of wetting solution is added to exceed the capacity of the start zone, allowing sample to move through the device in the manner of an ordinary liquid sample. In this way, the device can be stored prior to addition of the second volume to start the lateral flow.

After application of the sample capillarity draws liquid from the start zone, once saturated, into the next zones of the test strip. If a reagent zone is present the components of the assay dried on to it are rehydrated and carried with the liquid to the reaction zone. The components in the reaction zone are also rehydrated. Here, because the enzyme component(s) is immobilised, the analyte and all assay components are present in the same position, allowing the reaction to occur to produce a detectable signal.

The detectable signal might be either visible or measurable by an appropriate instrument like a change in absorption, fluorescence, luminescence, a change in pH, preferably a visible change in absorption.

In a preferred embodiment the detectable product formed by the enzyme reaction has a greatly reduced solubility in the solution of the reaction zone or a greater affinity for the matrix of the reaction zone than the substrate. This reduces movement of the product due to solvent flow, concentrating and depositing it at the reaction site and thus gaining sensitivity.

In a particular embodiment the matrix at and adjacent to the reaction zone is modified to give enhanced hydrophobic properties that further facilitate and localise deposition of the product.

The present invention further relates to a test kit at least comprising a test device according to the present invention. In addition, further aids, like wetting agent, standard solutions, a written protocol of the detection method, a standard colour chart etc. might be included. A stop solution might be included as a beneficial reagent for some enzyme reactions and/or applications.

The test device, test kit and method of the present invention are suitable for any enzyme assay in which an analyte can be detected by using an enzyme which is able to chemically (covalently) modify the analyte and thus directly or indirectly generates a detectable signal. Direct generation of a detectable signal means that after being chemically modified by the enzyme, the analyte itself generates a detectable signal. Indirect generation of a detectable signal means that another chemical(s) participating in the enzyme-mediated reaction involving the analyte or participating in a secondary reaction linked with the enzyme-mediated reaction is used to generate the detectable signal so that chemical(s) incorporated into the test strip acts as primary precursors for the detectable signal.

One example of an enzyme assay with indirect generation of a detectable signal is an assay in which the analyte is an essential co-factor or co-substrate for an enzyme which at the same time turns an additional substrate into a detectable product or a product which can be turned into an detectable product by an additional enzyme reaction or chemical reaction.

Examples of suitable enzyme assays are

the determination of glucose by the enzymes glucose dehydrogenase and diaphorase using a substrate formulation incorporating NAD and a suitable diaphorase substrate, for example NBT.

the determination of ATP by the methodology disclosed in WO 94/25619. Using the present disclosure, one skilled in the art could adapt this assay to the format of the present invention.

the determination of urea in biological fluids by the enzymes glutamate dehydrogenase, diaphorase and urease using a substrate formulation incorporating NADH, 2-oxoglutarate and a suitable diaphorase substrate, for example NBT.

the determination of serum cholesterol by the enzymes cholesterol oxidase and peroxidase using a substrate formulation incorporating suitable peroxidase substrates, for example 4-aminophenazone and phenol.

the determination of alcohol by the enzymes alcohol dehydrogenase and diaphorase using a substrate formulation incorporating NAD and a suitable diaphorase substrate, for example NBT.

the determination of carbon dioxide and/or bicarbonate in biological fluids by the enzymes phosphoenolpyruvate carboxylase, malate dehydrogenase and diaphorase using a substrate formulation incorporating phosphoenolpyruvate, oxaloacetate, NADH and a suitable diaphorase substrate, for example NBT.

the detection of biomass using a NAD(H) calorimetric assay

In the following suitable formats of an enzyme assay according to the present invention are exemplified with the NAD(H) calorimetric assay. This is a method for detecting the presence of biomass in a sample whereby the total pool of NAD(P) and NAD(P)H is measured as a marker for the presence of biomass by conversion of NAD(P) in the sample to NAD(P)H.

Nicotinamide adenine dinucleotides in the oxidised form, for example NAD and NADP, present in a sample can be converted to nicotinamide adenine dinucleotides in the reduced form, for example NADH and NADPH, by a suitable enzyme (E1) with its second substrate (S1).

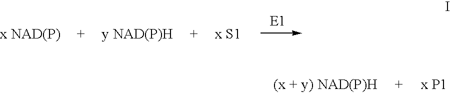

A suitable enzyme E1 is any enzyme that converts NAD(P) to NAD(P)H. In a preferred embodiment this enzyme is a dehydrogenase, for example glucose-6-phosphate dehydrogenase.

In a second step, both oxidised and reduced forms ((x+y) NAD(P)H according to scheme I) can be detected.

The detection is done by generating a detectable signal P2 using an additional enzymatic or chemical reaction, e.g. as depicted in reaction scheme II:

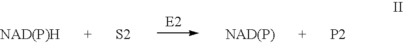

The signal from these reactions may be a change in colour, other spectral properties, fluorescence properties, luminescence or electrochemical potential.

In order to improve the sensitivity, both NAD(P) and NAD(P)H present in the sample can be recycled.

As could be seen from reaction scheme II, in terms of a chemical or enzymatic means of detection, NADH and NADPH are re-converted to NAD or NADP by reaction with a suitable donor molecule S2, either directly, via a suitable chemical mediator or via an enzyme catalysed reaction. Reduction of this donor, the second product of the dehydrogenase or the reduced form of the nicotinamide adenine dinucleotide produced leads to a signal P2 used as an indicator of the presence of biomass.

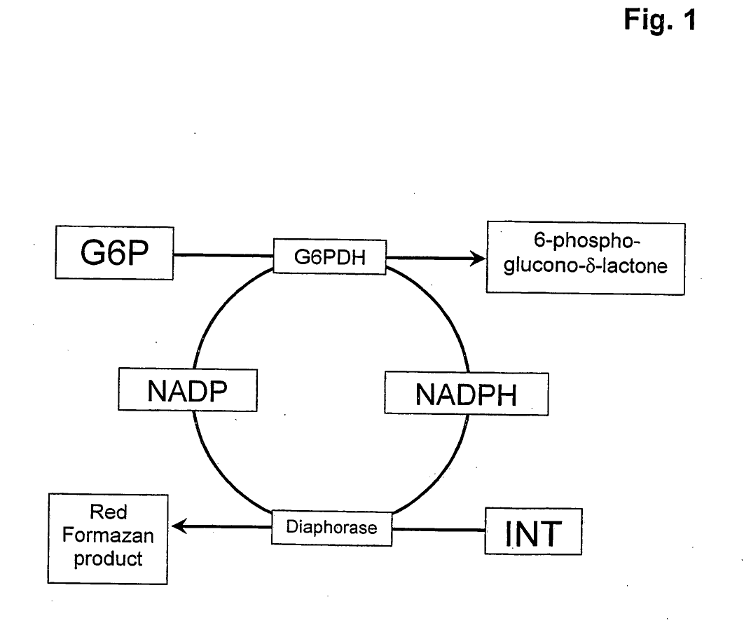

The NAD(P) generated in this reaction is then recycled into NAD(P)H again by using the same enzyme E1 that was used to originally convert the NAD(P) present in the sample into NAD(P)H. As a consequence a simple cycling system is generated which improves the sensitivity of the assay and is especially suitable for the detection of low amounts of biomass. To make this cycling system work properly it is necessary to provide an excess of the enzyme substrates S1 and S2. A scheme of the cycling reaction is shown in FIG. 1.

In one preferred format of the NAD(H) assay, the start zone, composed of an absorbent paper, holds a dried detergent extractant, the reagent zone, also composed of absorbent paper or of glass fibre, contains the substrates for the enzymes used in the assay, while the enzymes themselves are immobilised on the reaction zone, composed of a nitrocellulose membrane with a high protein binding capacity. Preferred reagents for the assay are DTAB (Dodecyl Trimethyl Ammonium Bromide) as detergent extractant on the start zone, the enzymes glucose dehydrogenase and diaphorase immobilised at the reaction zone and the corresponding substrates NBT and glucose dried onto the reagent zone.

Alternatively, DTAB can be incorporated into the substrate reagent dried on to the reagent zone.

Liquid sample applied to the start zone moves by capillary action into the reagent zone, where the enzyme substrates are rehydrated and move with the sample to the enzymes immobilised at the reaction zone. In the presence of the analytes NAD, NADH, NADP and/or NADPH the yellow NBT is converted to the dark blue formazan salt by the action of the enzymes and their substrates, producing a colour change visible by eye. The reduced solubility of the formazan product acts to concentrate it near the reaction site, giving a more intense colour. High sensitivity is achieved because the two enzymes recycle NAD(P) and NAD(P)H, producing many coloured molecules for each analyte molecule present in the sample, providing substrates are supplied from the reagent zone.

In other embodiments, other tetrazolium compounds or diaphorase substrates may be substituted for NBT to produce a readily detectable product with NAD(P)H and diaphorase. In another embodiment the enzyme diaphorase may be replaced by appropriate chemical reagents, for example Meldola's Blue and NBT, which can produce a readily detectable product in the presence of NAD(P)H.

To enable the measurement of two important parameters e.g. to monitor sample hygiene by a single device using the same sample, reagents that produce a colorimetric reaction with protein may be immobilised on the reaction strip at a separate location to the enzymes and substrates used to detect NAD(P) and NAD(P)H.

Assay of protein and NAD in the same sample provides additional sensitivity and information regarding the composition of the sample. As the reagent for detecting protein, those necessary for detection according to the various protein detecting methods, such as biuret reaction method, Lowry method, Coomassie dying method, BCA method and ninhydrin reaction method, are usable in the method of the invention. The reagent for detecting protein is preferably selected from the group of non-octahalogenated sulfophthaleines, desirably from the group consisting of phenol sulfonephthaleins and cresol sulfonephthaleins. Suitable reagents are for instance bromophenol blue, bromocresol green, bromocresol purple, bromophenol red, bromothymol blue, and bromochlorophenol.

It can be conceived that the same principles described for the detection of biomass using the NAD(H) calorimetric assay can also be applied to other enzyme assays, such that the substrate and other reagents for the assay are dried in pads on to the device upstream, in relation to the lateral flow of solution across the device, of the immobilised enzyme but at or downstream of the point of application or capture of sample.

It can be also be conceived that variations in the design of the device according to FIG. 2 can be utilised in order to facilitate incorporation of more than one such assay in a given device or enzyme assay in combination with other types of assay such as immunoassay or protein assay in a given device. In particular variations in the design can be conceived such that lateral flow from the sample zone of the device could occur in more than one direction to separated reaction zones of the respective assays (bi- or multi-directional lateral flow).

Compared to enzyme assays according to the state of the art, the lateral flow enzyme assay according to the present invention bears several advantages.

When an assay is performed with the previous simple test strip format using liquid reagent formulations in bottles, it involves single drops of substrate formulation and enzyme formulation dispensed onto a pad using dropper bottles. The typical drop size is approximately 30 μl of each solution and this is constrained, by the fluid dynamics of the bottles and solutions, from conveniently being reduced further without the use, for example, of volumetric micropipettes, which are typically used only by trained laboratory workers. In the equivalent assay, using the device format in the invention, with pre-dried reagents, only 4% or less of the enzyme in this formulation is needed. This represents a significant reduction in materials cost compared to the conventional liquid test format.

In addition, the test format according to the present invention

allows separate deployment of otherwise incompatible components independently dried on to the device in stable reagent formulations.

means that there is no requirement for any solution other than that incorporating the sample, such that the use of the device to perform an assay would require no more than one liquid addition step. Since all assay components are present on the same device, only one item is necessary to perform the assay, rather than several reagent bottles required for a conventional liquid test.

enables sampling, by use of the sampling pad, by wiping said sample pad across a surface or by dipping the sample pad into a liquid, without direct effect of the sampling action on the other reagents and substrates deployed elsewhere on the device. For example, in the case of sampling by dipping the sample pad into a liquid, the risk of loss of substrate reagents by elution into the bulk sample is low in relation to formats not using the lateral flow principle

enables sequential processing “steps” for the sample (for example: the sequence of extraction; mixing with substrate; enzyme reaction; product formation and deposition), such that the sole liquid addition initiates and results in suitable and even sequential rehydration and mixing of the reagents necessary for the assay. This simplifies the operation for the end-user.

does not require further steps or manipulations of the device by the user once the sample is added (examples: no filters to be removed; no washing steps).

requires relatively low amounts of components, especially enzyme components, in relation to a conventional liquid phase assay. This is especially important where enzymes are often the highest cost components in the assay.

prevents false background cross-reactions in storage, caused by minute amounts of contaminants present in one or more reagents of the assay, because the reagents are separated into two or more separate locations and also because the reagents are stored in the dry state.

reduces assay background, because importantly, during the assay, capillary flow draws away unreacted soluble assay components from the area of colour development—especially the precursors of the signal product. In this way, background effects of the non-specific conversion of precursor to product are reduced in comparison to a similar static (non-capillary flow) assay in which the soluble components are present indefinitely.

allows reading of the product on the reaction zone by eye and/or instrument reader.

allows high volume manufacture

allows a robust construction suitable for field use.

Without further elaboration, it is believed that one skilled in the art can, using the preceding description, utilise the present invention to its fullest extent. The preferred specific embodiments and examples are, therefore, to be construed as merely illustrative, and not limitative to the remainder of the disclosure in any way whatsoever.

The entire disclosures of all applications, patents, and publications cited above and below, and of corresponding application EP 01121820.3, filed Sep. 11, 2001, are hereby incorporated by reference.

Abbreviations used in figures, tables and elsewhere have the following meanings:

ATP adenosine-5′-triphosphate

DTAB dodecyl trimethyl ammonium bromide

NAD nicotinamide adenine dinucleotide (oxidised form)

NAD(P) nicotinamide adenine dinucleotide and nicotinamide adenine phosphate (oxidised form)

NAD(P)H nicotinamide adenine dinucleotide and nicotinamide adenine phosphate (reduced form)

NADH nicotinamide adenine dinucleotide (reduced form)

NADP nicotinamide adenine dinucleotide phosphate (oxidised form)

NADPH nicotinamide adenine dinucleotide phosphate (reduced form)

NBT nitro blue tetrazolium

### EXAMPLES

### Example 1

### Device for Assaying NAD

In an example of this invention a device, comprising a nitrocellulose reaction strip, paper sample pad, reagent pad and wick is constructed as shown in FIG. 2. Devices are based on the Millipore SR membrane, which comprises a nitrocellulose membrane on an inert plastic support. Self-adhesive portions on the plastic support allow attachment of suitable paper strips to act as sample pad, reagent pad and wick. Devices are constructed as 20 cm wide strips which are cut into individual devices with a width of 5 mm. Reagent pads are soaked in a solution of substrate formulation comprising 450 mM glucose, 2 mg/ml NBT, 10 mM citrate and then dried prior to assembly.

To prepare the strips for use 1 μl of enzyme formulation, comprising 200 mM Tris, 3 mg/ml diaphorase, 8 mg/ml glucose dehydrogenase is added to the nitrocellulose portion of the strip and allowed to dry at 20° C. for 10 minutes. In this configuration enzyme and substrate formulations are kept separate, improving the long term stability of the device. In addition each 5 mm width of reagent pad contains the equivalent of approximately 20 μl of substrate, ensuring that the immobilised enzyme on the reaction strip has substrate for sustained reaction time course. Significant savings are achieved compared to a conventional liquid formulation because the amount of enzyme used is much lower and enzymes are typically the most expensive chemical component of this type of assay.

The assay can be demonstrated by the addition of a solution containing 15 pmoles of NAD to the sample pad. The sample moves into the reagent pad where the enzyme substrates are rehydrated and carried with the sample into the nitrocellulose reaction pad. Here NAD, assay enzymes and substrates react to produce purple formazan product visible by eye within 5 minutes at 20° C., indicating that this device is capable of detecting NAD in solution. When the experiment is repeated using a distilled water control instead of an NAD solution as sample, no purple colour is formed after 10 minutes at 20° C.

### Example 2

### Device for Assaying Lactate

In a second example of this invention a device is constructed in the same manner as described in example 1 to detect the biologically important compound lactate. In this example the enzyme formulation consists of 400 mM Tris pH 8.0, 5 mg/ml diaphorase, 7.3 mg/ml lactate dehydrogenase and is dried onto the nitrocellulose portion of the strip as described in example 1. Reagent pads are prepared by soaking in a solution of 100 mM NAD, 2.5 mg/ml NBT and dried prior to assembly.

The assay can be demonstrated by the addition of a solution containing 1 μmole of lithium lactate to the sample pad. The assay proceeds as described in example 1. The lactate, enzymes and substrates react to produce purple formazan product visible by eye within 5 minutes at 20° C., indicating that this device is capable of detecting lactate in solution. When the experiment is repeated using a distilled water control instead of a lactate solution no purple colour is formed after 10 minutes at 20° C.

### Example 3

### Reduced Background Colour Formation

The previous NAD assay using liquid reagent formulations in bottles added to a pad on a test strip is susceptible to false background colour formation with time due to a light-induced interaction between NBT and the assay enzymes. As an experiment to assess this effect with the device of the invention, two devices prepared as described, in example 1 above are activated by the addition of 60 μl of distilled water to the sample pad. One device is kept in the dark for 5 minutes at 20° C., the other in ambient room light for 5 minutes at 20° C., which is sufficient to produce a light-induced background reaction in an equivalent liquid based test conducted on a sample pad in the previous configuration. No background colour formation is observed, even when the strips are left for an indefinite period in these lighting conditions indicating that this format is far less susceptible to interference by ambient room light and other such false background effects, due to the capillary migration of excess of substrate and other reactants beyond the location of the enzymes.

### Example 4

### Detection of Bacteria

To demonstrate the use of the device to detect bacteria, 30 μl of a broth culture of E.coli NovaBlue (Novagen, Catalogue No.69009-3) containing 7.2×10⁷ colony forming units/ml (determined by total aerobic microbiological plate count) is added to the sample pad of a strip prepared as described in example 1. To activate the device 30 μl of an extractant solution consisting of 0.3% w/v DTAB in water is added to the sample pad, providing sufficient volume for the liquid sample to move through the device by capillary action. The extractant lyses the bacteria present on the sample pad, allowing intracellular NAD(P) and NAD(P)H to move to the reaction pad via the reagent pad with the mobile sample phase. Purple colour indicating the presence of NAD(P)(H), equivalent to a “dirty” result in a hygiene monitoring assay, is obtained within 5 minutes. When the experiment is repeated using sterile broth containing no bacteria, no colour formation is observed.

### Example 5

### Hygiene Testing

To demonstrate the use of the device to sample the hygiene of surfaces, a test stainless steel surface, typical of those used in the commercial food preparation and manufacturing sectors, is smeared with an extract of fresh turkey mince and allowed to dry. A 30 μl drop of distilled water is added to the sample pad of a strip prepared as described in the second example, and the sample pad drawn across the surface to transfer any debris from the surface to the pad. Care is taken not to touch the reaction pad or reagent pad and to ensure that only the sample pad comes into contact with the test surface. The assay is initiated by the addition of a 30 μl drop of extractant solution, containing 0.3% w/v DTAB, to the pad, which releases any NAD(P) or NAD(P)H molecules present in debris in the sample pad, and allows liquid sample to move to the reaction pad via the reagent pad. Purple colour is formed on the reaction pad within 5 minutes, indicating the presence of biological material on the test surface. When the experiment is repeated without wiping the sample pad over the dirty test surface, no colour formation is observed.

1. Method for determining the presence of an analyte in a sample comprising:
a) providing a test device incorporating a lateral flow test strip comprising a dry matrix material capable of transporting a liquid therealong by capillarity and having at least a start zone for receiving said sample and a reaction zone having at least one kind of enzyme immobilised therein;
b) contacting the start zone with said sample thereby causing a liquid phase to move through said test strip;
c) determining a detectable signal at the reaction zone directly or indirectly caused by covalent modification of the analyte by the enzymes at the reaction zone

2. Method according to claim 1, characterised in that the test strip provided in step a) comprises an additional reagent zone.

3. Method according to claim 1 characterised in that the test strip provided in step a) comprises additional dried reagents in the start zone and/or the reagent zone and/or the reaction zone.

4. Method according to claim 1, characterised in that the liquid flow generated in step b) is enhanced by a wick on the test strip.

5. Method according to claim 1, characterised in that the detectable signal is generated by a product of an enzymatic reaction which is visible by eye and barely soluble.

6. Method according to claim 1, characterised in that the detectable signal in step c) is generated by using a recycling enzyme reaction.

7. Lateral flow test device for determining the presence of an analyte in a sample incorporating a test strip comprising a dry matrix material capable of transporting a liquid therealong by capillarity and having at least a start zone for receiving said sample and a reaction zone having at least one kind of enzyme immobilised therein whereby the enzymes and optional additional reagents on the test strip are suitable for covalently modifying the analyte and thus directly or indirectly generating a detectable signal.

8. Lateral flow test device according to claim 7, characterised in that the test strip further comprises a solid support on which is attached the dry matrix material.

9. Lateral flow test device according to claim 7, characterised in that the test strip further comprises a reagent zone containing dried reagents.

10. Lateral flow test device according to claim 7, characterised in that the test strip comprises a start zone containing a dried detergent extractant, a reagent zone containing one or more dried enzyme substrates and/or cofactors and a reaction zone containing at least one kind of immobilised enzyme.

11. Lateral flow test device according to claim 7, characterised in that the test strip further comprises a wick.

12. Lateral flow test device according to claim 7, characterised in that it further comprises a housing.

13. Lateral flow test device according to claim 7, characterised in that the reaction and/or reagent zones are protected by a transparent film to allow ease of handling and prevent contamination while allowing viewing of the signal formed at the reaction zone

14. Lateral flow test device according to claim 7, characterised in that the test strip comprises a start zone containing DTAB (Dodecyl Trimethyl Ammonium Bromide) as detergent extractant, a reagent zone containing the substrates NBT and glucose and a reaction zone containing the immobilised enzymes glucose dehydrogenase and diaphorase.

15. Use of a lateral flow test device according to claim 7 in an enzyme assay.

16. Use of a lateral flow test device according to claim 14 to test surfaces.

17. Use of a lateral flow test device according to claim 14 to test liquids.

18. Enzyme assay test kit comprising a lateral flow test device according to claim 7.
